(19) United States
(12) Patent Application Publication
(10) Pub. No.: US 2022/0248697 A1
(43) Pub. Date: Aug. 11, 2022

(54) AN ORGANIC, NATURAL ANTIMICROBIAL PRESERVATIVE FOR MEAT PRODUCTS

(71) Applicants: Venkata Satya Sarveswara Sairam Kuchimanchi, Secunderabad (IN); Vaishnavi Kuchimanchi, Secunderabad (IN)

(72) Inventors: Venkata Satya Sarveswara Sairam Kuchimanchi, Secunderabad (IN); Vaishnavi Kuchimanchi, Secunderabad (IN)

(21) Appl. No.: 17/432,332

(22) PCT Filed: Aug. 12, 2019
(86) PCT No.: PCT/IN2019/050591

§ 371 Date: Aug. 19, 2021

(30) Foreign Application Priority Data
Apr. 25, 2019 (IN) 201941016364

(51) Int. Cl. A23B 4/20; A23L 3/3508; C12P 39/00; C12P 9/00

(52) U.S. Cl. CPC A23B 4/20; A23L 3/3508; A23V 2002/00; C12P 9/00; C12P 39/00

(57) ABSTRACT

The present invention relates to an “Organic, Natural Antimicrobial preservative for Meat products” comprising of natural organic salts (majorly lactates and acetates) which are a completely safe, organic, natural ingredients for preventing spoilage of fresh meats and sausages under refrigeration and imparts an enhanced shelf life to the meat products. The present organic preservative is produced by a two-step microbial fermentation of reducing sugars extracted from sweet potato and cassava starches using plantarum” for the first anaerobic fermentation step followed by the second “aerobic fermentation” step by a bacterial “Lactobacillus consortium comprising of Saccharomyces cerevisiae and Acetobacter aceti followed by downstream purification steps. It imparts a long term preservation and shelf life to the meat products for a period of more than 20-25 days for fresh brined meat and more than 6-8 months for processed and cured, refrigerated meats.

### FIELD OF THE INVENTION

The present invention relates to an organic product with antimicrobial properties, produced naturally through a two-step microbial fermentation of carbohydrates from sweet potato and cassava sources using bacterial strains of Lactobacillus plantarum, Saccharomyces cerevisiae, and Acetobacter aceti. This product prevents spoilage of fresh meats and sausages under refrigeration and imparts an enhanced shelf life to the meat products. It is a safe and natural preservative which is good for gut health and is capable of effecting antimicrobial activity for a long time in meat products treated with it.

### BACKGROUND OF THE INVENTION

Avoiding spoilage during storage and transport of meat products, sausages, and fish products has always been a great concern for meat industry professionals. Spoilage of food products can be caused by a wide range of physical, chemical, enzymatic, and microbial reactions. Examples of most widely used chemical food preservatives that inhibit microbes include benzoates, nitrites, ascorbates, and sorbates (01). But as per the food safety regulations, these chemical preservatives cannot be used above the recommended dosage level of 0.05-0.1%. Also, there is always one or the other health risk associated with the use of synthetic chemical additives and hence natural preservatives are preferred both by sellers and consumers.

Plant essential oils, as well as similar compounds, have been considered as promising natural antimicrobials (02). The presence of fat, carbohydrate, protein, salt, and pH generally influence the effectiveness of these agents in foods. Their antimicrobial strength is also reduced in foods with low water activity (02).

Originally added to change or improve taste, spices and herbs can also be used to enhance the shelf life of food products by inhibiting foodborne pathogenic bacterial and fungal species. Usually, compounds with phenolic groups are considered to be effective antimicrobials but they are generally more inhibitory against Gram-positive than Gram-negative bacteria (03, 04, 05).

The applicant's “Organic, Natural Antimicrobial preservative for Meat products” is an effective antimicrobial product with proven benefits in controlling microbial growth and proliferation for a broad range of deleterious food spoilage microbes, and helps extend the shelf life of all type of meat products without influencing the tenderness, textural properties and flavor of the meat products.

It can be a better alternative to chemical preservatives as it doesn't have any toxic effects on gut health and is a safe natural alternative while retaining the advantages of shelf life enhancement and imparting desired aroma and flavors.

### SUMMARY OF THE INVENTION

The present invention relates to an “Organic, Natural Antimicrobial preservative for Meat products”, which is a completely organic, natural ingredient for controlling unwanted microbial load in meat products. This product prevents spoilage of fresh meats and sausages under refrigeration and imparts an enhanced shelf life to meat products. It is a safe and natural preservative which is good for gut health and is capable of effecting antimicrobial activity for a long time in meat products treated with it.

In one of the embodiments of the present invention, the active ingredient in “Organic, Natural Antimicrobial preservative for Meat products” is a combination of naturally produced Organic salts, including lactates and acetates that have antimicrobial properties along with flavor improving efficiency for the preserved meat product.

Another embodiment of the present invention relates to a process of producing “Organic, Natural Antimicrobial preservative for Meat products”, produced through a two-step microbial fermentation of carbohydrates from sweet potato and cassava sources to obtain natural organic salts (majorly lactates and acetates) as preservative components using bacterial strains of Lactobacillus plantarum, Saccharomyces cerevisiae, and Acetobacter aceti by downstream processing steps including filtration and concentration.

In one of the embodiment, the “microbial source” used for fermentative production of this product includes three bacterial strains namely Saccharomyces cerevisiae NCIM 3594, Acetobacter aceti NCIM 2094 and Lactobacillus plantarum NCIM 2084, which were modified by way of strain improvement through medium optimization experiments for product yield enhancement at the ‘in house R&D section’ of Prathista Industries Limited.

In one of the embodiments of the present invention, “Lactobacillus plantarum” is the effector microbe for the first anaerobic fermentation step for lactic acid production, followed by the second “aerobic fermentation” step for acetic acid production by a bacterial consortium comprising of Saccharomyces cerevisiae and Acetobacter aceti.

In other embodiment of the present invention, a diluted solution [10.0% (v/v) in demineralized water] of “Organic, Natural Antimicrobial preservative” is applied @ 10 mL per Kg of meat weight during the brining process of fresh ground meat before vacuum packaging and refrigeration of fresh ground meat/beef/pork preparations.

In another embodiment of the present invention during the preparation of cured meats such as sliced, cooked, and boneless hams; sliced cooked loaves; snack sticks; jerky; barbecued meats (pulled pork or beef); poultry rolls and breasts; roast beef etc., the “Organic, Natural Antimicrobial preservative” [10.0% (v/v) in demineralized water] applied @ 0.5% v/w of meat weight during the hydration process, has the potential to replace the chemical flavoring and curing agents like nitrates/nitrites without much impacting the expected meat texture, appearance and flavor of the cured meat from controls while imparting a long term preservation and shelf life to the meat products by way of retention of bacteriostatic effect and effective control in microbial spoilage for a period of more than 20-25 days for fresh brined meat and more than 6-8 months for processed, cured, and refrigerated meats.

### BRIEF DESCRIPTION OF DRAWINGS

FIG. 1: Comparative profile of Listeria sp. growth on fresh ground beef upon application of different treatments. Control: Ground beef treated with 0.9% v/w saline solution; T 1: “Organic, Natural Antimicrobial preservative” @ 0.5% v/w of meat weight; T2: “Organic, Natural Antimicrobial preservative” @ 1.0% v/w of meat weight; T3: “Organic, Natural Antimicrobial preservative” @ 1.5% v/w of meat weight. T4: “Organic, Natural Antimicrobial preservative” @ 2.0% v/w of meat weight.

FIG. 2: Aerobic plate count on Fresh chicken meat upon application of different treatments. Control: Chicken meat treated with 0.9% v/w saline solution; T1: “Organic, Natural Antimicrobial preservative” @ 0.5% v/w of meat weight; T2: “Organic, Natural Antimicrobial preservative” @ 1.0% v/w of meat weight; T3: “Organic, Natural Antimicrobial preservative” @ 1.5% v/w of meat weight.

FIG. 3: Flow chart of the process for Organic Anti-mold Bakery additive production.

### DETAILED DESCRIPTION OF THE INVENTION

### (A) Overview

The present invention relates to an “Organic, Natural Antimicrobial preservative for Meat products” which is a completely organic, natural ingredient for controlling unwanted microbial load in meat products. This product prevents spoilage of fresh meats and sausages under refrigeration and imparts an enhanced shelf life to meat products. It is a safe and natural preservative which is good for gut health and is capable of effecting antimicrobial activity for a long time in meat products treated with it.

In one of the embodiment of the present invention, the active ingredient in “Organic, Natural Antimicrobial preservative for Meat products” is a combination of naturally produced Organic salts, including lactates and acetates that have antimicrobial properties along with flavor improving efficiency for the preserved meat product. In one of its preferred embodiment, this product comprises of naturally produced lactic and acetic acids which impart antimicrobial properties, thereby inhibiting microbial growth on the treated food product (especially meat products), thus preventing spoilage without affecting its inherent flavor and texture.

The other embodiment of the present invention relates to a process of production of an “Organic, Natural Antimicrobial preservative for Meat products” produced through microbial fermentation of natural carbohydrate sources followed by downstream processing steps including filtration and concentration.

In this embodiment of the invention, the applicant's product is produced by microbial fermentation of reducing sugars extracted from sweet potato & cassava starches to obtain natural organic salts (majorly lactates and acetates) as preservative components. This product is produced by a two-step microbial fermentation process followed by downstream purification steps. “Lactobacillus plantarum” is the effector microbe for the first anaerobic fermentation step for lactic acid production, followed by the second “aerobic fermentation” step for acetic acid production by a bacterial consortium comprising of Saccharomyces cerevisiae and Acetobacter aceti.

In one of the embodiment, the “microbial source” used for fermentative production of this product includes three bacterial strains namely Saccharomyces cerevisiae NCIM 3594, Acetobacter aceti NCIM 2094, and Lactobacillus plantarum NCIM 2084, which were modified by way of strain improvement through medium optimization experiments for product yield enhancement at the ‘in house R&D section’ of Prathista Industries Limited.

In other embodiment of the present invention, a diluted solution [10.0% (v/v) in demineralized water] of “Organic, Natural Antimicrobial preservative” is applied @ 10 mL per Kg of meat weight during the brining process of fresh ground meat before vacuum packaging and refrigeration of fresh ground meat/beef/pork preparations.

In another embodiment of the present invention during the preparation of cured meats such as sliced, cooked, and boneless hams; sliced cooked loaves; snack sticks; jerky; barbecued meats (pulled pork or beef); poultry rolls and breasts; roast beef etc., the “Organic, Natural Antimicrobial preservative” [10.0% (v/v) in demineralized water] applied @ 0.5% v/w of meat weight during the hydration process, has the potential to replace the chemical flavoring and curing agents like nitrates/nitrites without much impacting the expected meat texture, appearance and flavor of the cured meat from controls while imparting a long term preservation and shelf life to the meat products by way of retention of bacteriostatic effect and effective control in microbial spoilage for a period of more than 20-25 days for fresh brined meat and more than 6-8 months for processed and cured, refrigerated meats.

The present invention is further explained by the following examples. However, the present invention is not limited to these examples in any manner. The following examples are intended to illustrate the working of disclosure and not intended to take restrictively to apply any limitations on the scope of the present invention. Those persons skilled in the art will understand that the equivalent substitutes to the specific substances described herein, or the corresponding improvements are considered to be within the scope of the invention.

### (B) Experimental Details & Results

This product is produced by a two-step microbial fermentation process—anaerobic fermentation and aerobic fermentation. All fermentation studies were carried out in stirred-tank jacketed Stainless Steel (S.S.) bioreactors of 25 L and 50 L capacity.

### Example 1

### (i) Upstream Processing

Anaerobic Fermentation:

The first step involves anaerobic fermentation for Lactates production using “Lactobacillus plantarum” as the effector microbe. The Lactobacillus strain used for this fermentation is a culturally improved bacterial strain modified by medium optimization experiments using the stock strain of Lactobacillus plantarum NCIM 2084. Fermentation was carried out on a synthetic medium with the following composition (% w/v):

| Components | % w/v |
|---|---|
| Glucose | 11-15% |
| Yeast extract | 1% |
| Potassium dihydrogen phosphate | 0.1% |
| Diammonium hydrogen phosphate | 0.2% |
| Manganese sulphate | 0.001% |
| Cobalt chloride | 0.001% |
| Magnesium sulphate | 0.001% |
| Sodium chloride | 0.001% |
| Ferrous sulphate | 0.0005% |

Medium (without glucose) was heat sterilized at 121° C. and 15 psi for 25 min. Glucose was autoclaved separately at 115° C. for 15 min and added aseptically to the rest of the medium.

The anaerobic fermentation process parameters were as follows:

| Parameter | Value | |
|---|---|---|
| pH | 6.5 ± 0.2 | |
| Temperature | 45 ± 2° | C. |
| Agitation (RPM) | 100 | |
| Nitrogen | 0.3 | L/min |

The pH was maintained using potassium carbonate (K₂CO₃). Temperature and pH were monitored using temperature and pH probe, respectively (Sartorius). Sterile nitrogen gas was flushed into the headspace of the reactor using a sterile 0.2 μm pore sized PTFE filter (Axiva® 200050 RI, AXIVA Sichem Biotech Pvt. Ltd., India), to maintain anaerobic condition throughout this fermentation step. The pre-sterilized fermentation medium in the bioreactor was inoculated @ 10% inoculum of 48 hrs grown static flask culture. The seed culture was prepared in 500 mL Erlenmeyer flasks, incubated at 45° C. under anaerobic conditions in Anaerobic S.S. jars with the help of Whitley Jar Gassing System (Don Whitley Scientific Limited, UK). After 84 to 90 h fermentation, complete glucose is consumed and lactic acid concentration of 10-12% is achieved in the fermented broth. Thereafter, the broth is subjected to a second fermentation step (aerobic fermentation) for acetate production.

### Aerobic Fermentation:

Acetate production is achieved using a microbial consortium comprising of two in-house improved strains namely Saccharomyces cerevisiae NCIM 3594 and Acetobacter aceti 2094 modified by medium optimization experiments. Fermented broth from 25 L fermenter is transferred to a 50 L fermenter containing an equal volume of pre-sterilized medium.

The aerobic fermentation medium composition (% w/v) was as follows:

| Components | % w/v |
|---|---|
| Glucose | 8-10% |
| Yeast extract | 0.5% |
| Potassium dihydrogen phosphate | 0.1% |
| Diammonium hydrogen phosphate | 0.2% |
| Manganese sulphate | 0.00015% |

Medium (without glucose) was heat sterilized at 121° C. and 15 psi for 25 min Glucose was autoclaved separately at 115° C. for 15 min and added aseptically to the rest of the medium.

The aerobic fermentation process parameters were as follows:

| Parameter | Value | |
|---|---|---|
| pH | 5.5 ± 0.2 | |
| Temperature | 30 ± 2° | C. |
| Agitation (RPM) | 100 | |
| Nitrogen | 0.5 | L/min |

The pH was maintained using potassium carbonate (K₂CO₃). Temperature and pH were monitored using temperature and pH probe, respectively (Sartorius). The pre-sterilized fermentation medium in the bioreactor was inoculated @ 10% inoculum of 24 hrs grown stirred flask culture. The seed culture was prepared in 500 mL Erlenmeyer flasks, incubated at 30° C. at 120 rpm.

### Example 2

### (ii) In-process Monitoring of Microbial Growth and Product Yield

Measurement of microbial growth during fermentation was done in terms of optical density, in 3-mL cuvettes, at a wavelength of 600 nm using UV-Vis spectrophotometer. For dry cell weight estimation, 10-15 mL of fermentation broth was centrifuged at 10,000 rpm for 10 min in a pre-weighed falcon tube and dried at 60° C. under vacuum till constant weight was achieved.

### Example 3

(iii) Downstream Processing and Product Recovery

Immediately upon surge of organic acids production, neutralization was done through dosing with pre-sterilized potassium carbonate slurry for the formation of a potassium-enriched organic acids based product. As the maximal production of organic acids and complete utilization of glucose is achieved within 70 h of aerobic fermentation step, a typical production batch is terminated between 60 to 72 h of fermentation. This is followed by filtration through 0.3 to 0.4-micron size cloth filters in a plate and frame filtration assembly. The filtered product is concentrated by evaporation at a high temperature of about 80° C. to achieve the desired product specification of the respective organic acids before packing. The upstream and downstream process has been depicted as a flow chart in FIG. 3.

### Example 4

### (iv) Shelf—Life Testing

The impact of the “Organic, Natural Antimicrobial preservative” on shelf-life of fresh, uncured meat was assessed in terms of the growth of bacterial pathogen Listeria sp. in samples treated with varying concentrations of the product over a period of 45 days at a storage temperature of 4° C. Samples treated with only 0.9% w/v saline solution served as ‘Control’.

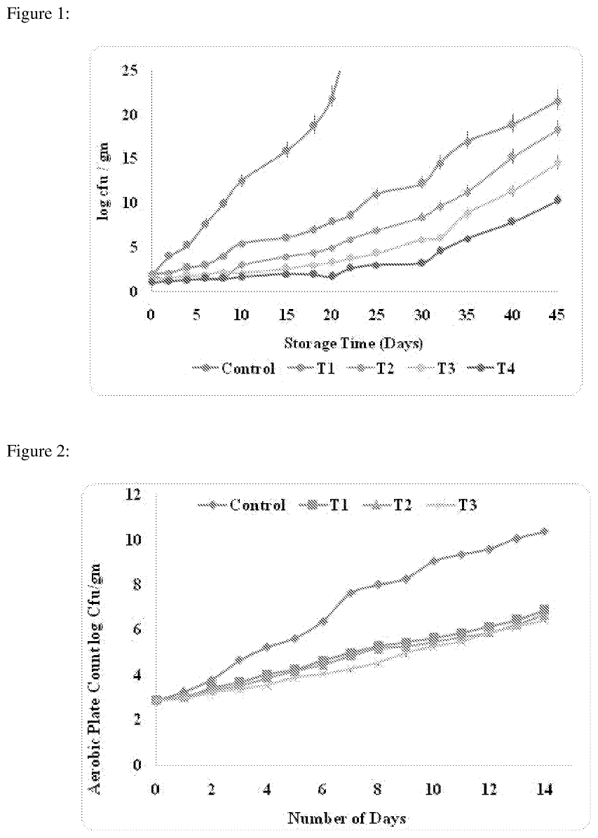

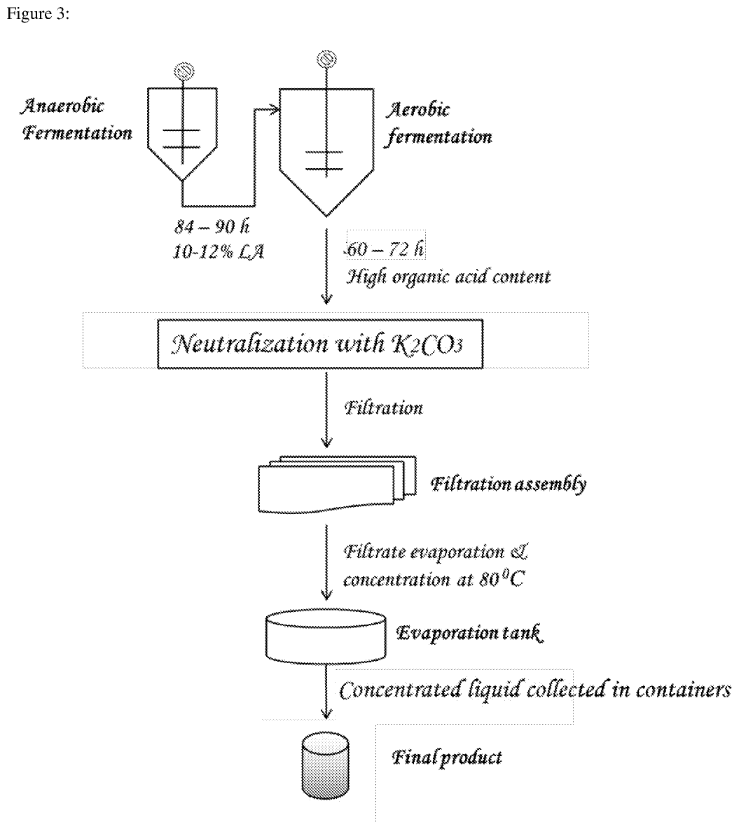

10 g samples of fresh, uncured meat (ground beef) and raw chicken breast were analyzed for microbial growth. It was observed that the ground beef sample containing “Organic, Natural Antimicrobial preservative” as additive @ 2.0% v/w could sustain for more than one month without significant increase in microbial load (FIG. 1), whereas the raw chicken meat sample treated @ 1.5% v/w of the additive prevented spoiling for up to 30 days (FIG. 2). In contrast, the ‘Control’ meat samples showed growth of pathogenic bacteria day 3 onwards.

### Industrial Applicability of the Invention

This invention has applicability as a natural, clean label, antimicrobial preservative for meat and fish products during storage and transportation.

### REFERENCES CITED

1) Anand, S. P. and Sati, N. (2013) Artificial preservatives and their harmful effects: looking toward nature for safer alternatives. International Journal of Pharmaceutical Sciences and Research, 4(7): 2496-2501.
2) Seow, Y. X., Yeo, C.R., Chung, H. L., and Yuk, H. G. (2014) Plant Essential Oils as Active Antimicrobial Agents. Critical Reviews in Food Science and Nutrition, 54 (5): 625-644.
3) Hammer, K. A., Carson, C. F., and Riley, T. V. (1999) J. Appl. Microbiol., 86(6):985.
4) Kalemba, D., Kunicka, A. (2003) Curr. Med. Chem., 10(10): 813.
5) Bagamboula C.F., Uyttendaele M., Debevere J. (2003) J. Food Prot., 6(4): 668.

1. An organic, natural antimicrobial preservative comprising of a combination of naturally produced organic salts, including lactates and acetates produced by microbial fermentation of carbohydrates, which prevents spoilage of fresh meats and sausages under refrigeration and imparts an enhanced shelf life along with flavor improving efficiency for the preserved meat products.

2. A process of production of the organic, natural antimicrobial preservative as claimed in claim 1, wherein it is produced naturally through a two-step microbial fermentation of carbohydrates from sweet potato and cassava sources using bacterial strains of Lactobacillus plantarum, Saccharomyces cerevisiae, and Acetobacter aceti by downstream processing steps including filtration and concentration.

3. The organic, natural antimicrobial preservative as claimed in claim 1, wherein the microbial source used for fermentative production includes three bacterial strains namely Saccharomyces cerevisiae NCIM 3594, Acetobacter aceti NCIM 2094 and Lactobacillus plantarum NCIM 2084, which were modified by way of strain improvement through medium optimization.

4. The organic, natural antimicrobial preservative as claimed in claim 3, wherein Lactobacillus plantarum is the effector microbe for the first anaerobic fermentation step for lactic acid production, followed by the second aerobic fermentation step for acetic acid production by a bacterial consortium comprising of Saccharomyces cerevisiae and Acetobacter aceti.

5. The process as claimed in claim 2, wherein the first step involves anaerobic fermentation for Lactates production using “Lactobacillus plantarum” as the effector microbe on a synthetic medium with the following composition (% w/v): glucose 11-15%, yeast extract 1%, potassium dihydrogen phosphate 0.1%, diammonium hydrogen phosphate 0.2%, manganese sulphate 0.001%, cobalt chloride 0.001%, magnesium sulphate 0.001%, sodium chloride 0.001% and ferrous sulphate 0.0005%.

6. The process as claimed in claim 5, wherein anaerobic fermentation is carried out at pH 6.0±0.2, temperature 45±2° C., agitation 100 rpm and nitrogen 0.3 L/min. for 84 to 90 hours.

7. The process as claimed in claim 2, wherein second fermentation step (aerobic fermentation) for acetate production by a bacterial consortium comprising of two improved strains Saccharomyces cerevisiae NCIM 3594 and Acetobacter aceti 2094 is carried out on the synthetic medium comprising of the following composition (% w/v): glucose 8-10%, yeast extract 0.5%, potassium dihydrogen phosphate 0.1%, diammonium hydrogen phosphate 0.2% and manganese sulphate 0.00015% at pH 5.50±0.2, temperature 30±2° C., agitation at 100 rpm, aeration 0.5 L/min.

8. The process as claimed in claim 7, wherein the aerobic fermentation is carried out for 60-72 hours.

9. The process as claimed in claim 5, wherein medium (without glucose) is heat sterilized at 121° C. and 15 psi for 25 min; glucose is autoclaved separately at 115° C. for 15 min and added aseptically to rest of the medium.

10. The process as claimed in claim 2, to 9, wherein it is carried out in stirred-tank jacketed Stainless Steel (S.S.) bioreactors of 25 L and 50 L capacity.

11. The process as claimed in claim 2, wherein neutralization of the organic acids produced through fermentation is done by potassium carbonate slurry.

12. The process as claimed in claim 2, wherein filtration is carried out the through 0.3 to 0.4-micron size cloth filters in a plate and frame filtration assembly.

13. The process as claimed in claim 2, wherein the filtered product is concentrated by evaporation at 80° C.

14. The organic, natural antimicrobial preservative as claimed in claim 1, wherein diluted solution [10.0% (v/v) in demineralized water] of the said preservative is applied @ 10 mL per Kg of meat weight during the brining process of fresh ground meat before vacuum packaging and refrigeration of fresh ground meat/beef/pork preparations and [10.0% (v/v) in demineralized water] applied @ 0.5% v/w of meat weight during the hydration process to impart long term preservation and shelf life to meat products for a period of more than 20-25 days for fresh brined meat and more than 6-8 months for processed and cured, refrigerated meats.
